(19) United States
(12) Patent Application Publication
(10) Pub. No.: US 2014/0026916 A1
(43) Pub. Date: Jan. 30, 2014

(54) Method for Reducing Marine Pollution Using Polyhydroxyalkanoate Microbeads

(71) Applicant: College of William and Mary, Williamsburg, VA (US)

(72) Inventors: Kirk J. Havens, Plainview, VA (US); Donna Marie Bilkovic, Gloucester Point, VA (US); David M. Stanhope, Hayes, VA (US); Kory T. Angstadt, Gloucester, VA (US)

(73) Assignee: College of William and Mary, Williamsburg, VA (US)

(21) Appl. No.: 13/950,493

(22) Filed: Jul. 25, 2013

Related U.S. Application Data
(60) Provisional application No. 61/675,410, filed on Jul. 25, 2012.

(51) Int. Cl. A61K 8/85; A61Q 11/00; A61Q 19/10

(52) U.S. Cl. CPC A61K 8/85; A61Q 19/10; A61Q 11/00
USPC 132/200

(57) ABSTRACT

Herein we describe a method for the reduction of pollution in aquatic systems by incorporating polyhydroxyalkanoate microbeads into personal care formulations such as exfoliants, cosmetics, and toothpaste. Suitable polyhydroxyalkanoate microbeads are biodegradable, have an average size of less than 400 microns, and sink rapidly in aquatic environments.

### CROSS-REFERENCE TO RELATED APPLICATIONS

This patent application claims the benefit of and priority to U.S. Provisional Patent Application Ser. No. 61/675,410 filed Jul. 25, 2012, the entire contents of which are herein incorporated by reference.

### STATEMENT REGARDING FEDERALLY SPONSORED RESEARCH OR DEVELOPMENT

Not applicable

### FIELD OF INVENTION

The present invention relates to methods for reducing plastic pollution from personal care products by incorporating suitable biodegradable polyhydroxyalkanoate microbeads into personal care formulations.

### BACKGROUND

Plastic debris in the world's oceans and estuaries was reported in the 1970's (Carpenter et al. 1972; Carpenter and Smith 1972) and gained increased scrutiny over the succeeding decades (Andrady 2011). The annual global production of plastic has been reported between 230 to 245 million tonnes (Browne et al. 2011, Cole et al. 2011; Andrady 2011) and due to the low cost, bio-inertness, longevity, and resultant multitude of uses, plastic production is likely to continue to increase. In addition, with around 50% of the global population residing with 100 km of the coast and populations increasing in coastal areas, the likelihood of increased introduction of plastics in the marine environment is high (Kershaw and Leslie 2012).

The fragmentation of plastic and the introduction of microplastic beads has become a significant concern in the world's marine environments. Microplastics are defined as plastic fragments or particles ranging in size from a few μm to 500 μm (Andrady 2011), though Arthur and others (2009) defined microparticles as less than 333 μm. Microplastics result from the breakdown of larger plastic debris, typically polyethylene and polypropylene, over a prolonged period of time through biological, chemical, and physical processes or through the manufacture of microspheres or microbeads (Costa et al. 2010; Roy et al. 2011).

Manufactured microbeads of non-degradable plastics such as polyethylene, polypropylene, and polystyrene are used in personal care products such as makeup, suncare, oral care, and skin care products, as well as household cleanser products and industrial sand blasting (Gregory 1996; Fendall and Sewell 2009). Kershaw and Leslie (2012) reported microbeads in widely used consumer products, with microbeads ranging in size from about 20 μm to 70 μm. Fendall and Sewell (2009) reported the mode size of microbeads in three out of four facial cleanser brands was less than 100 μm. Liebezeit and Dubaish (2012) reported polyethylene microbeads in commercial shower gels and peelings of about 1% to 4% by weight.

Microbeads used in cosmetics and cleansers ultimately end up in the wastewater stream. Typically, the fine and very fine screens from wastewater systems do not adequately remove microplastics smaller than 200 μm (EPA 2003), thus allowing many plastic microbeads from commercial cosmetics and toiletries to pass through or potentially accumulate in the treatment plant digestion tanks or flocculate with other sewage particulate matter. One potential consequence of synthetic solids accumulation in digestion tanks is the rejection of biosolids for reuse as a soil amendment (EPA 2003). Microbeads are of particular concern because the relative density of polyethylene and polypropylene is less than 1.0, resulting in the potential for microbeads to float through stormwater, sewage, and septic systems and down tributaries to estuarine and marine ecosystems (Gregory 1996; Browne et al. 2011).

Once microbeads enter the aquatic environment, their buoyancy, size, and longevity within the water column can affect the aquatic food web. Microbeads have the potential to be ingested by both pelagic and benthic organisms. Laboratory studies have shown uptake by marine biota of microplastics in size ranges from 2 μm to 70 μm (see review by Cole et al. 2011). Microbeads of sizes less than 200 μm can be ingested by filter-feeders, detritivores, deposit feeders, and planktivores (Brown et al. 2007; Fendal and Sewell, 2009). Browne and others (2007) reported on the accumulation of polystyrene microbeads in the gut of mussels (Mytilus edulis). Farrell and Nelson (2013) reported on the transfer of microbeads from the tissue of mussels to the haemolymph of the crabs feeding on them. Microplastics or their derivates have also been reported in fish (Beorger et al. 2010) and baleen whales (Fossi et al. 2012). Microbeads made from plastics such as polypropylene, and particularly polyethylene, can also sorb and concentrate organic contaminants and transport these compounds to marine organisms at various tropic levels (Teuten et al. 2009; Andrady 2011).

A review of historical plankton samples shows microplastics in the water column have increased significantly over the last 40 years, trending with the increase in the global production of plastic (Browne et al. 2007). However, Kershaw and Leslie (2012) suggest that estimates of the quantities of microplastics may be underestimates due to the capacity of the terrestrial system to serve as a temporary repository for microplastics and the large mesh sizes of marine sampling nets that miss microplastics in the water column smaller than 330 μm. In a study of beaches in the Lower Saxonian Wadden Sea, Liebezeit and Dubaish (2012) concluded that cosmetic-derived microbeads are a major source of microplastics.

One solution is to use microbeads that are degradable, including micobeads that are biodegradable. For example, biopolymers such as polylactic acid (“PLA”) are degradable in terrestrial composting systems, and in the human body, but unfortunately are not routinely degradable in aquatic systems. PLA microbeads have been used as encapsulating agents for pharmaceuticals, and also as an FDA-approved cosmetic product (Sculptra™, available from Sanofi Aventis) that is injected into deep layers of the skin to improve facial appearance as collagen deposits form around the microbeads. In our experience, we have found that polycaprolactone (“PCL”) can biodegrade in marine environments. However, the low heat-resistance and high softness/stickiness of PCL makes PCL microbeads undesirable in cosmetic and toiletries formulations.

Alternatively, naturally occurring or derived exfoliants are made from materials such as almond shells and jojoba oil. Ceramic microspheres made from amorphous magnesium silicate are commercially available and used in cosmetics formulations.

However, many consumer products companies still use plastic microbeads that are not biodegradable. It is an object of this invention to reduce aquatic pollution by providing polymer microbeads for personal care products with improved cost/performance characteristics and low potential to adversely impact the environment.

### BRIEF SUMMARY OF THE INVENTION

Herein we describe a method for reducing aquatic pollution comprising applying a personal care formulation to a human body, and rinsing said formulation off the body with water, wherein said formulation comprises polyhydroxyalkanoate (“PHA”) microbeads have an average size of less than 400 microns; and wherein said polyhydroxyalkanoate microbeads sink rapidly in aquatic environments. The rinse water ultimately drains into an aquatic environment selected from the group consisting of a wastewater treatment facility, a septic system, a stormwater system, a sewer system, a surface waterbody, and a groundwater aquatic environment, wherein a surface waterbody includes oceans, bays, lakes, reservoirs, rivers, streams, and ponds.

Herein we also describe a method for reducing aquatic pollution comprising formulating a product useful for cleaning or protecting human body surfaces, and directing users to apply the product to body surfaces, wherein said product comprises PHA microbeads have an average size of less than 400 microns; and wherein said PHA microbeads sink rapidly in aquatic environments.

In one aspect of the invention, PHA microbeads are used to facilitate exfoliation by applying PHA microbeads to the skin as part of a cosmetic formulation.

In another aspect of the invention, PHA microbeads are used to facilitate cleaning of teeth by applying PHA microbeads to the teeth and gums as part of a toothpaste formulation.

In another aspect of the invention, PHA microbeads are preferred relative to microbeads of non-degradable polymers, or other degradable polymers, because the short residence time of PHA microbeads reduces the likelihood for toxic persistent organic pollutants to sorb to the microbeads when in the aquatic environment, thereby minimizing risk to food webs and human health.

In some embodiments of the invention, the PHA microbeads are spherical, or nearly so, and useful for enhancing the feel of a lotion, and/or useful for reducing wrinkle lines.

In other embodiments of the invention, the PHA microbeads have reduced circularity, and are useful for cleaning body surfaces. These abrasive PHA microbeads can have varying degrees of hardness depending on the desired application, and typically have hardnesses in the range 1-4 on the Mohs Hardness Scale.

In preferred embodiments, the PHA microbeads, once rinsed and subsequently released into aquatic environments, sink to the bottom of the aquatic environment. In some embodiments, the PHA microbeads are then sequestered by sediment-dwelling organisms such as clamworms, and buried or incorporated into structures such as tubes. Once sequestered, the PHA microbeads are not easily re-suspended, and can quickly break down and are less likely to sorb persistent organic pollutants or be ingested by aquatic organisms.

In preferred embodiments, at least 25% of the PHA microbeads will sink towards the bottom at a rate of at least 0.25 inches per minute when placed in a surface waterbody.

PHA microbeads can be incorporated into personal care formulations suitable for products such as makeup, toothpaste, exfoliants, and sunscreen.

### BRIEF DESCRIPTION OF THE FIGURES

FIG. 1 is an image of PHA microspheres taken at a magnification of 350×.

FIG. 2 shows an image of PHA microspheres taken at a magnification of 2800×, and includes measurements of the diameter of selected PHA microbeads.

FIG. 3 shows an image of PHA microspheres taken at a magnification of 2000×.

FIG. 4 shows a microscope image of PHA microbeads having an approximate size range of 100 μm to 180 μm, and modest circularity, with utility as an abrasive cleaning particle in a personal care formulation.

FIG. 5 shows a series of microscope images of sunken 180-300 micron PHA microbeads in a beaker containing seawater and sediment. Images taken after two days, five days, and nine days (FIGS. 5A, 5B, and 5C respectively) show progressive degradation and sequestration within the sediment.

FIG. 6 shows a microscope image of PHA microbeads, some of which were dyed (microbeads at the left of the image) and some of which were not dyed (microbeads at the right of the image).

FIG. 7 shows photographic images of settling tests conducted on PHA microbeads according to the Standard Methods for the Examination of Water and Wastewater, Section 2540F. FIG. 7A is a photograph of PHA microbeads sized from approximately 297 μm to 425 μm in an Imhoff cone during the test procedure, while FIG. 7B is a photograph of PHA microbeads sized from approximately 45 μm to 100 μm in an Imhoff cone during the test procedure.

FIG. 8 shows a microscope image of PHA microbeads of low circularity that floated when subjected to a settling test conducted according to the Standard Methods for the Examination of Water and Wastewater, Section 2540F.

### DETAILED DESCRIPTION OF THE INVENTION

A personal care formulation, as used herein, refers to a product intended for application to a human body that is useful for personal hygiene and/or beautification. Personal care products include, but are not limited to: lip balm, cleansing pads, colognes, cotton pads, deodorant, eye liner, lip gloss, lipstick, lotion, makeup, mouthwash, pomade, perfumes, shampoo, conditioner, shaving cream, skin cream, sunscreen, wet wipes, and toothpaste.

PHA microbeads have a size defined by their area-equivalent diameter (ISO 9276-6:2008(E) section 7), also called Equivalent Circle Diameter (“ECD”, ASTM F1877-05 Section 11.3.2). The mean ECD of a PHA microbead population is calculated as the average of respective ECDs of each PHA microbead (excluding microbeads) having ECD of below 10 microns). The microbead particles have a mean ECD from for example, 10 μm, 50 μm, 75 μm, or 100 μm to, for example, 1000 μm, 500 μm, 350 μm, or 250 μm. In preferred embodiments for use in cleaning applications, the PHA microbeads have a mean ECD of about 30 μm to about 300 μm. When PHA microspheres are used, the preferred size range is from about 10 μm to about 100 μm.

The term “circularity” refers to the shape of microbead particles, and is a quantitative, 2-dimensional image analysis shape description as measured according to ISO 9276-6:2008(E) section 8.2. Circularity is sometimes described in literature as being the difference between a particle's shape and a perfect sphere. Circularity values range from 0 to 1, where a circularity of 1 describes a perfectly spherical particles or disc particle as measured in a two dimensional image. In some embodiments of the invention, PHA microbeads having high circularity (for example, PHA microspheres) are desirable, whereas in other examples for which more abrasive particles are desired, the preferred circularity of PHA microbeads can be below 0.5.

Depending on the particular personal care application, the PHA microbeads should be sufficiently hard to provide good cleaning/cleansing performance while providing good surface safety and/or skin feel acceptability. The hardness of the PHA microbeads may be expressed according to the MOHS hardness scale. The PHA microbeads may have a MOHS hardness between 0.5 and 4, and preferably, between 1 and 3. The MOHS hardness scale is an internationally recognized scale for measuring the hardness of a compound versus a compound of known hardness, see Encyclopedia of Chemical Technology, Kirk-Othmer, 4th Edition Vol 1, page 18 or Lide, D. R (ed) CRC Handbook of Chemistry and Physics, 73 rd edition, Boca Raton, Fla.: The Rubber Company, 1992-1993.

PHA polymers are true biopolymers, produced in nature by bacterial fermentation of sugar and lipids. They are linear polyesters, and more than 150 different monomers can be combined within this family to give polymers with a wide variety of properties. Some common PHA polymers include poly-3-hydroxybutyrate, poly-4-hydroxybutyrate, polyhydroxyvalerate, poly-3-hydroxyhexanoate, and co-polymers thereof, including poly(3-hydroxybutyrate-co-4-hydroxybutyrate), also known as P(3HB-co-4HB); poly(3-hydroxybutyrate-co-valerate; also known as PHBV; poly(3-hydroxybutyrate-co-3-hydroxyhexanoate), also known as PHBH. The Nodax class of PHA polymers, originally developed at Procter and Gamble and currently sold commercially by Meredian Inc., includes PHBH and also other PHA copolymers that contain 3-hydroxybutyrate monomer units as well as other 3-hydroxyalkanoate monomer units having longer side chains.

In applications wherein harder PHA microbeads are desired, it is advantageous to use PHA microbeads containing a high percentage of hydroxybutyrate monomer units, either 3-hydroxybutyrate or 4-hydroxybutrate. These hydroxybutyrate monomers units contain four carbons, whereas hydroxyvalerate units contain five carbons, and hydroxyhexanoate units contain six carbons. In order to enhance the hardness of PHA microbeads, the constituent monomer units of the PHA should be comprised of at least 90% hydroxybutyrate monomer units.

PHA microbeads can be produced by a number of methods known in the prior art for making plastic microbeads. When microbeads are spherical, or nearly so (when circularity is greater than 0.95), they are known as microspheres. PHA microspheres are a class of PHA microbeads, and are particularly useful in cosmetic formulations to improve the feel of the formulated product and to reduce the appearance of wrinkles by optical scattering.

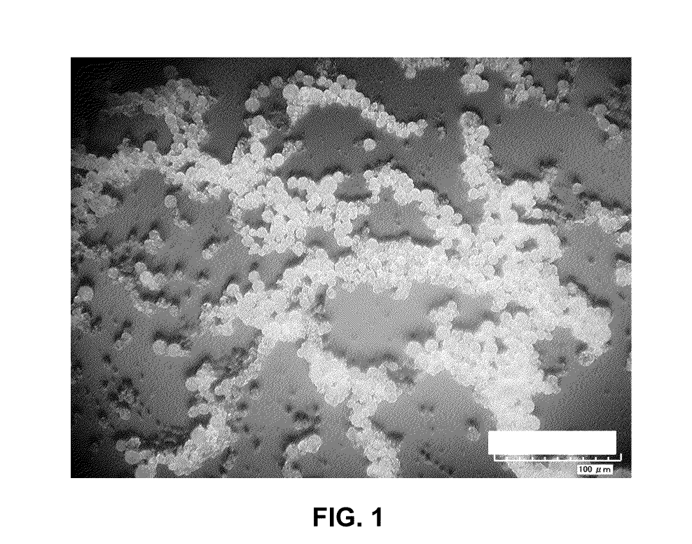

PHA microspheres can be produced using methods known in the art for making PHA or other polymer microspheres. For example, PHA microspheres of high circularity can be produced via spray drying of a solution of PHA in a suitable solvent. Alternatively, we have created PHA microspheres by emulsion solvent evaporation using dichloromethane to dissolve the PHA polymer. The PHA solution is emulsified with an emulsifying agent in an aqueous solution using a high-speed homogenizer. Additional water is added, and the emulsion is stirred, causing PHA microspheres to form as the organic solvent evaporates. The PHA microspheres can be collected by filtration. See FIGS. 1-3 for images of PHA microspheres produced using this method.

By altering the stirring speed, the ratio of organic solvent to water, and the concentration of emulsifying agent, the size and particle distribution of the resulting PHA microbeads can be adjusted. Pigments or dyes can be added to impart color and/or opacity to the microbeads. Entrapment of active agents or other additives can also be achieved by adding them to the mixture, for example, prior to forming the emulsion. For example, alpha hydroxyl acids can be entrapped or adsorbed onto the microspheres.

Numerous organic solvents can be used to produce PHA microspheres using the solvent evaporation technique, provided the starting PHA is miscible in the organic solvent. For example, chloroform and dichloromethane can be used.

One or more additives useful for enhancing the emulsion can be used, including well-known emulsifying agents such as polyvinyl alcohol, gelatin, polysorbate, methyl cellulose, and hydroxypropylmethyl cellulose.

PHA microspheres can be incorporated into personal care formulations using techniques known in the art. The PHA microspheres are free-flowing powders.

A PHA microsphere will be transparent when its refractive index is identical to the refractive index of the medium it is in. This can be advantageous when tactile properties of microspheres are desired without diminishing gloss (e.g., in a lip gloss application).

In typical cosmetic formulations, PHA microspheres can be incorporated at concentrations ranging from about 0.1% w/w to 25% w/w, with so-called créme to powder formulations typically containing a very high concentration of PHA microspheres.

Absorbent PHA microbeads can provide a different feel relative to, for example, polyethylene beads, and can help reduce visible signs of sweating.

In some embodiments, PHA microspheres are coated with active agents, for example, alpha hydroxyl acids.

PHA microbeads of low circularity, which are particularly useful in exfoliant and cleansing formulations, can be produced using numerous methods, including those known in the art for producing microbeads of low circularity from other polymers. For example, PHA microbeads can be produced directly from the PHA biosynthetic production process by isolating PHA from the mixture. In other embodiments, PHA microbeads can be produced from PHA pellets by grinding, blending, or other mechanical degradation methods.

In some preferred embodiments for personal care formulations used for cleaning body surfaces, the polyhydroxyalkanoate microbeads have an average circularity of greater than 0.35 and less than 0.75.

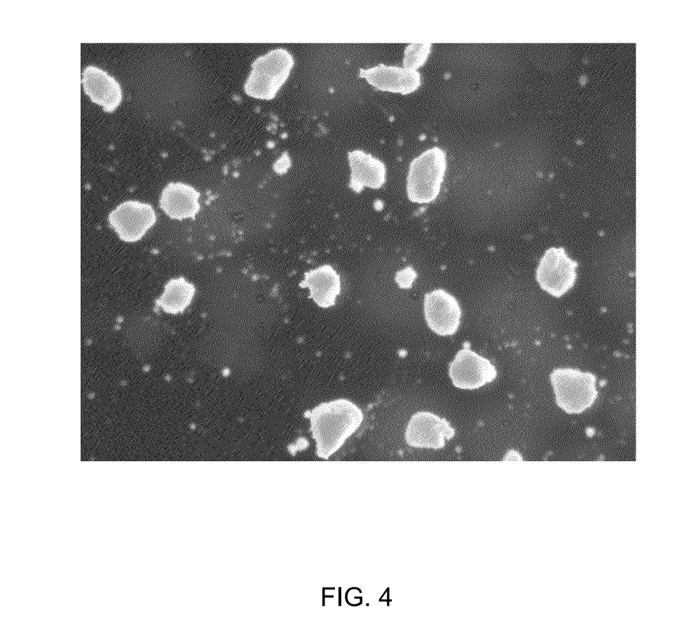

PHA microbeads can be sieved or otherwise purified by size to produce PHA microbead compositions having reasonably narrow size ranges; for example, see FIG. 4 for an image of PHA microbeads having a size range 100 μm to 180 μm. For example, careful application of sieves using known protocols can allow isolation of microbeads falling within a reasonably narrow size range, for example, we have prepared microbead fractions wherein most of the microbeads fell within the particle size range: 45 μm to 100 μm, or 100 μm to 180 μm, or 180 μm to 250 μm, or 250 μm to 297 μm, or 297 μm to 425 μm.

In preferred embodiments, personal care formulations containing PHA microbeads have PHA microbead average sizes of less than 425 microns, preferably less than 400 microns. Depending on the particular application, the lower limit of microbead size range can be, for example, 10 microns, 20 microns, 50 microns, 100 microns, 200 microns, or any other appropriate size less than 400 microns. Many PHA microbead product samples will contain some very small particles, particularly after storage and handling, even if, for example, the microbeads are described as having a size range of 100 to 300 microns. Suitable PHA microbead compositions useful according to the methods of the invention have median particle sizes of up to 425 microns, and in preferred embodiments, the median particle size of PHA microbeads is greater than 10 microns, and less than 400 microns.

In some preferred embodiments for personal care formulations used for cleaning body surfaces (e.g., toothpaste, exfoliating scrub), the polyhydroxyalkanoate microbeads have an average circularity of greater than 0.35 and less than 0.75.

In contrast to non-degradable polymers such as polypropylene and polyethylene which float and thus can remain suspended in the water column and travel large distances (Browne et al. 2011; Liebezeit and Dubaish 2012), most PHA polymers have a relative density of around 1.30 and will tend to sink, depending on the size and shape of the particles. Negative buoyancy reduces the potential distance PHA microbeads travel in aquatic or groundwater systems and can allow for quick biodegradation in septic tanks, wastewater treatment plants, and tributaries before the microplastics reach estuarine and marine ecosystems. In contrast, non-degradable microbeads are nearly impossible to remove in any quantity from the aquatic system and once they are incorporated into food webs cannot be removed. This has far-reaching economic ramifications in terms of ecosystem services such as fisheries, food supply, water quality, and human health.

PHA is known to biodegrade in aquatic and other environments (e.g., see Jendrossek and Handrick 2002 for a review). For example, we have demonstrated that injection-molded PHA panels degrade in marine environments (see U.S. Patent Application Publication No. 20120160351 A1). Importantly, PHA biodegrades in such environments, as a purely chemical or mechanical mechanism of degradation in aqueous environments would render PHA microbeads unsuitable for use in many personal care products that use a water-based formulation.

PHA microbeads when used according to the methods of the invention tend to sink, whether in fresh water or salt water environments. This property can be tested according to the Standard Methods for the Examination of Water and Wastewater, Section 2540F (“Settleable Solids”), American Public Health Association (1999). The procedure is provided below.

1. General Discussion. Settleable solids in surface and saline waters as well as domestic and industrial wastes may be determined and reported on either a volume (mL/L) or a weight (mg/L) basis.

2. Apparatus. The volumetric test requires only an Imhoff cone. The gravimetric test requires all the apparatus listed in Section 2540D.2 and a glass vessel with a minimum diameter of 9 cm.

3. Procedure. Fill an Imhoff cone to the 1-L mark with a well-mixed sample. Settle for 45 minutes, gently agitate sample near the sides of the cone with a rod or by spinning, and allow to settle for an additional 15 minutes. Record volume of settleable solids in the cone as milliliters per liter. If the settled matter contains pockets of liquid between large settled particles, estimate volume of these and subtract from volume of settled solids. The practical lower limit of measurement depends on sample composition and generally is in the range of 0.1 to 1.0 mL/L.

Where a separation of settleable and floating materials occurs, do not estimate the floating material as settleable matter.

In one embodiment, at least 95% of the volume of PHA microbeads in a personal care formulation must sink below the water surface (although not necessarily all the way to the bottom) during the one-hour time period of the Settleable Solids Standard Method for the Examination of Water and Wastewater, Section 2540F. In another embodiment, at least 25% of the volume of PHA microbeads in a personal care formulation settles to the bottom of an Imhoff cone during the one-hour time period of the the Settleable Solids Standard Method for the Examination of Water and Wastewater. In certain embodiments of the invention that are particularly useful for cleaning applications, at least 95% of the volume of PHA microbeads microbeads in a personal care formulation settle to the bottom of an Imhoff cone during the one-hour time period of the the Settleable Solids Standard Method for the Examination of Water and Wastewater.

Typically, the settling rate of PHA microbeads depends on a number of factors, including but not limited to the chemical composition of the PHA microbeads, the shape of the microbeads, and the size of the microbeads. In general, PHA microbeads that are larger and more spherical tend to sink more quickly, while PHA microbeads with very low circularity tend to remain on the surface. For example, PHA microbeads created by applying high shear forces (e.g., with a blender) to PHA pellets tended to have low circularity, and a significant fraction of such PHA microbeads remained on the surface during the Settleable Solids Standard Method for the Examination of Water and Wastewater. These PHA microbeads are not well-suited to personal care formulations, as they tend to be too abrasive, and are likely to be more persistent in the environment since they do not readily sink to the bottom of an aquatic system.

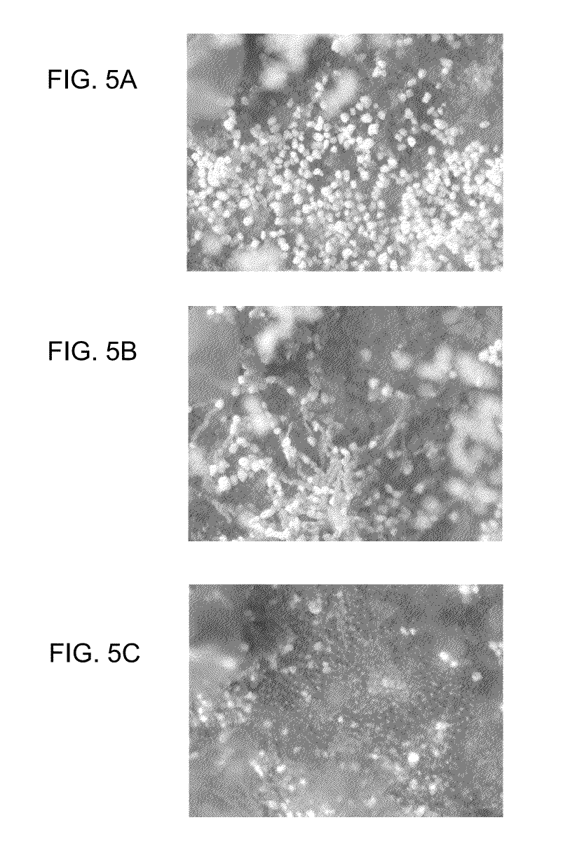

Benthic tube building worms and amphipods (and some insect larvae in freshwater systems), gather, bury, process, and in some cases cement (with mucus) sand, silt, and other particles found on the bottom sediments. As discussed above, PHA microbeads tend to sink. Once on the bottom, the PHA microbeads can be incorporated into tubes alongside sand or silt particles, retained in biodeposits, or buried in the sediment (for example, see FIGS. 5A, 5B, and 5C), reducing the opportunity for PHA microbead re-suspension, and accelerating rapid microbial degradation into benign monomers and oligomers. In contrast, polyethylene and polypropylene have relative densities less than 1.0 and float, thus greatly increasing their availability to aquatic organisms in the water column. Polyethylene and similar nondegradable plastics can sorb persistent organic pollutants, and since these plastics float they can end up washed up in concentration on intertidal beaches. Benthic organisms that attempt to utilize these particles can be exposed to toxics over very long time periods. Selective sequestering of PHA by bottom dwelling organisms is an advantageous property for rapid biodegradation of PHA microbeads.

Personal care formulations useful for the methods of the invention may comprise the aforementioned PHA microbeads or combinations of said PHA microbeads. Suitable personal care formulations may comprise from 0.1%, 0.3%, 0.5%, or 1% to 20%, 10%, 7%, or 4%, by weight of the total composition, of said PHA microbeads.

A personal care formulation useful for the methods of the invention may be, for example, a skin care, lip care, anti-perspirant, deodorant, cosmetic, oral care, or hair care product, and can be applied to the face, neck, hands, arms, mouth, hair, or any other part of the body. The personal care formulation may be used as, for example, a moisturizer, conditioner, toothpaste, anti-aging compound, skin lightener, sunscreen, sunless tanner, shave preparation, lipstick, foundation, mascara, after-shave, and combinations thereof.

The personal care formulation may involve a wide variety of forms. Non-limiting examples include simple solutions (e.g., water or oil-based), dispersions, and emulsions. The personal care formulation may be substantially anhydrous. “Substantially anhydrous” means that the composition comprises no more than about 1%, 0.5%, or, 0% water. The personal care compositions may be fluid or solid (gels, sticks, flowable solids, amorphous materials). In certain embodiments, the personal care composition is in the form of an emulsion. Emulsions may be generally classified as having a continuous aqueous phase (e.g., oil-in-water and water-in-oil-in-water) or a continuous oil phase (e.g., water-in-oil and oil-in-water-in-oil).

In select embodiments, the personal care formulation may be in a form comprising at least one discrete, visually distinct first phase and at least one discrete, visually distinct second phase. For purposes of these select embodiments, “visually distinct” means that the phases can be separately seen by the human eye as distinctly separate regions (i.e., not emulsions or dispersions of particles).

The personal care formulation may comprise a carrier. Carriers may be selected for various stability, aesthetics, and/or compatibility with other materials present in the personal care formulation. Suitable carriers include water and/or water soluble solvents. The personal care formulation may comprise from about 1% to about 95% by weight of water and/or water-equivalent solvent. The composition may comprise from about 1%, 3%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55% ,50%, 45%, 40%, 35%, 30%, 25%, 20%, 15%, 10%, or 5% water and/or a water-equivalent solvent. “Water-equivalent solvent” refers to a compound which has a similar ability as water to solubilize a material. Suitable water-equivalent solvents include monohydric alcohols, dihydric alcohols, polyhydric alcohols, glycerol, glycols, polyalkylene glycols such as polyethylene glycol, and mixtures thereof. Particularly suitable solvents, include lower aliphatic alcohols such as ethanol, propanol, butanol, isopropanol; diols such as 1,2-propanediol, 1,3-propanediol, butanediol, pentanediol, hexanediol, heptanediol, decanediol; glycerin; water, and mixtures thereof. In certain embodiments, the personal care composition comprises water, diols, glycerin, and combinations thereof.

Suitable carriers also include oils. The personal care composition may comprise from about 1% to about 95% by weight of one or more oils. The composition may comprise from about 1%, 3%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, or 5% of one or more oils. Oils may be used to solubilize, disperse, or carry materials that are not suitable for water or water-equivalent solvents. Suitable oils include silicones, hydrocarbons, esters, fatty amides, ethers, and mixtures thereof. Oils may be fluid at room temperature. However, certain personal care product forms (i.e., solid or semi-solid stick) may require non-fluid oils. The oils may be volatile or nonvolatile. “Non-volatile” means a material that exhibits a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm of mercury at 20° C. Volatile oils may be used to provide a lighter feel when a heavy, greasy film is undesirable.

Suitable oils include volatile oils. In certain embodiments, the volatile oils may have a viscosity ranging from about 0.5 to about 5 centistokes at 25° C. Volatile oils may be used to promote more rapid drying of the skin care composition after it is applied to skin. Nonvolatile oils are also suitable for use in the composition. Nonvolatile oils are often used for emolliency and protective properties. Nonvolatile oils preferably may have a viscosity ranging from about 5 to about 800,000 cst (or greater) or from about 20 to about 200,000 cst.

Suitable silicone oils include polysiloxanes. Polylsiloxanes may have a viscosity of from about 0.5 to about 1,000,000 centistokes at 25° C.

The personal care composition may comprise an emulsifier. An emulsifier is particularly suitable when the composition is in the form of an emulsion or if immiscible materials are being combined. The skin care composition may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.”

The personal care formulation may comprise a structuring agent. Structuring agents may be used to increase viscosity, thicken, solidify, or provide solid or crystalline structure to the personal care composition. The structuring agent may be used to suspend or disperse the abrasive particles. Structuring agents are typically grouped based on solubility, dispersibility, or phase compatibility. Examples of aqueous or water structuring agents include polymeric agents, natural or synthetic gums, polysaccharides, and the like. In one embodiment, the composition may comprises from about 0.0001%, 0.001%, 0.01%, 0.05%, 0.1%, 0.5%, 1%, 2%, 3%, 5% to about 25%, 20%, 10%, 7%, 5%, 4%, or 2%, by weight of the composition, of one or more structuring agents.

Polysaccharides and gums may be used as aqueous phase thickening agents. Examples of such polysaccharides and gums include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, ammonium alginate, calcium alginate, calcium carrageenan, carnitine, carrageenan, guar gum, guar hydroxypropyltrimonium chloride, hyaluroinic acid, hydroxypropyl chitosan, hydroxypropyl guar, karaya gum, kelp, locust bean gum, natto gum, potassium alginate, potassium carrageenan, propylene glycol alginate, sodium carboyxmethyl dextran, sodium carrageenan, tragacanth gum, and mixtures thereof. Suitable polysaccharides include alkyl hydroxyalkyl cellulose ethers such as cetyl hydroxyethylcellulose, which is the ether of cetyl alcohol and hydroxyethylcellulose. Other useful polysaccharides include scleroglucans comprising a linear chain of (1-3) linked glucose units with a (1-6) linked glucose every three units, a commercially available example of which is Clearogel™ CS11 from M.M.P., Inc.

Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof.

The personal care compositions may comprise one or more optional components to provide an efficacious and/or consumer desirable product. For example, the composition can include other actives or agents. For instance, suitable optional actives and agents may include an active or agent selected from a group consisting of sugar amines, vitamins, oil control agents, photosterols, hexamidine compounds, tightening agents, anti-wrinkle actives, anti-atrophy actives, flavonoids, N-acyl amino acid compounds, retinoids, peptides, particulate materials, UV actives, photostabilizers, anti-cellulite agents, desquamation actives, anti-acne actives, anti-oxidants, radical scavengers, conditioning agents, anti-inflammatory agents, tanning actives, skin lightening agents, botanical extracts, antimicrobial actives, antifungal actives, antibacterial actives, antiperspirant actives, sensates, preservatives, anti-dandruff actives, substantivity polymers, detersive surfactants, and combinations thereof.

Any other suitable optional component can also be included in the personal care composition of the present invention, such as those ingredients that are conventionally used in given product types. The Personal Care Product Council's International Cosmetic Ingredient Dictionary and Handbook, Thirteenth Edition, 2010, describes a wide variety of nonlimiting functional materials that can be added to the composition herein. Examples of these functional classes include, but are not limited to: abrasives, absorbents, fragrances, anti-acne agents, anti-caking agents, antifoaming agents, antimicrobial agents (e.g., iodopropyl butylcarbamate), antifungal agents, antioxidants, binders, buffering agents, bulking agents, chelating agents, colorants, cosmetic astringents, cosmetic biocides, denaturants, drug astringents, external analgesics, film formers, opacifying agents, pH adjusters, plant derivatives, plant extracts, plant tissue extracts, plant seed extracts, plant oils, botanicals, botanical extracts, preservatives, propellants, reducing agents, sebum control agents, sequestrants, skin bleaching agents, skin-conditioning agents (e.g. humectants and occlusive agents), and skin protectants. Other suitable optional person care ingredients include materials listed in paragraphs 513-839 of U.S Patent Application No. 2010/0112100.

### EXAMPLES

The examples that follow are intended in no way to limit the scope of this invention but are provided to illustrate the methods of the present invention. Many other embodiments of this invention will be apparent to one skilled in the art.

### Example 1

### Production of PHA Microbeads

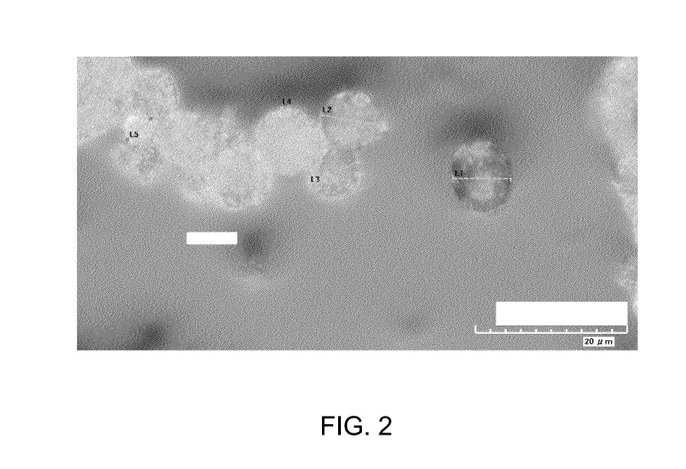

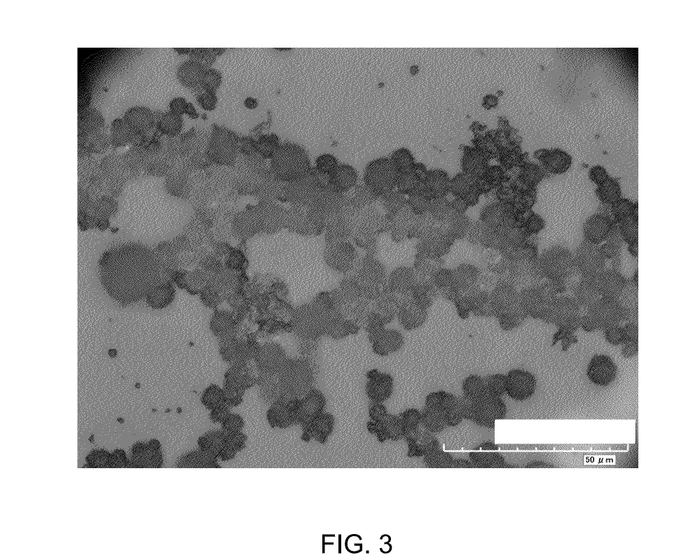

Approximately 1 g of PHA pellets (obtained from Metabolix in Lowell, Mass.) was dissolved in 75 mL dichloromethane, and non-soluble particulates were removed by filtration. Approximately 10 mL of the PHA/dichloromethane solution was mixed with 30 mL of a 2% solution of methyl cellulose in water, and the mixture was homogenized for 3 minutes using a Virtis homogenizer. To the resulting emulsion was added 40 mL water, and the mixture was stirred for approximately 45 minutes to evaporate dichloromethane and produce PHA microbeads. The PHA microbeads were separated via centrifugation, then collected and dried. Representative images of the PHA microbeads, taken with a HIROX KH-7700 digital microscope, are shown in FIGS. 1-3. FIG. 1 shows a HIROX image of PHA microbeads at a magnification of 350×. FIG. 2 shows a HIROX image of PHA microbeads at a magnification of 2800×, and includes measurements of the diameter of selected PHA microbeads. FIG. 3 shows a HIROX image of PHA microbeads at a magnification of 2000×. The size of the particles marked L2 through L5 in FIG. 2 ranges from 4.1 μm to 9.2 μm.

PHA microbeads were made using variations of the experimental conditions above, altering the relative concentrations of water and dichloromethane, the concentration of methyl cellulose, and the stirring speed during the evaporation phase, yielding a range of sizes of microbeads.

### Example 2

### Formulation of PHA Microbeads

PHA microbeads can be incorporated into an exfoliating scrub as follows:
Part A. 1. Propylene Glycol 30.00
2. Glycerin, 30.00
3. Methyl Gluceth-20, 33.30
4. Acrylates/C10-30 Alkyl Acrylate Crosspolymer, Carbopol®* Ultrez 20 Polymer 1.00
Part B. 5. Fragrance, 0.50 Fragrance
6. PHA microbeads, 3.80
7. Sodium Magnesium Aluminium Silicate, Liquibead 10PC Red D2, 0.50
8. Euterpe Oleracea Pulp Powder, 0.10
9. Phenoxyethanol (and) Ethylhexylglycerin, Euxyl® PE 9010, 0.80 Preservative

### Procedure:

1. PART A: Mix Propylene Glycol, Glycerin and Methyl Gluceth-20 in a suitable mixing vessel.
2. Disperse Carbopol®* Ultrez 20 Polymer by sprinkling on the surface of PART A with mixing (800-1,200 rpm). Mix until the polymer has completely dispersed, up to three hours.
3. PART B: Add PART B ingredients to batch one at a time, in order with mixing at low speed. Mix batch until uniform.

### Example 3

### Buoyancy Testing of PHA Microbeads

PHA powder was obtained from Shenzhen Ecomann Biotechnology Co. (Shenzhen, China), and sieved to provide PHA microbeads having the following size ranges: 0-45 microns, 45-100 microns, 100-180 microns, 180-250 microns, 250-297 microns, and 297-425 microns. These isolated PHA microbeads can be used in personal care formulations, and can also be combined, for example, combining two of the smaller fractions yields PHA microbeads having a particle size range of approximately 45 microns to 180 microns.

PHA pellets were obtained from Metabolix, and blended at high speeds to create microbeads having a low average circularity. The PHA microbeads were sieved to provide PHA microbeads having the following size ranges: 0-45 microns, 45-100 microns, 100-180 microns, 180-250 microns, 250-297 microns, and 297-425 microns.

The two sets of PHA microbeads (classified below as Ecomann and Mirel) were tested to determine how quickly the microbeads sank in water. Approximately 1 g of the PHA microbeads were added with moderate stirring to approximately one liter of water in a beaker with a water depth of 14 cm. The recorded times in Table 1 below represents the time range from the first microbead reaching the bottom of the beaker to the last microbead reaching the bottom of the beaker. The test was stopped after three minutes, and a recorded time of incomplete (“INC”) was given if not all microbeads had sunk to the bottom of the beaker.

TABLE 1

| | Time for Ecomann | Time for Mirel |
|---|---|---|
| Microbead Size | PHA Microbeads to | PHA Microbeads to |
| Range (μm) | Sink (seconds) | Sink (seconds) |
| 45-100 | 35 to INC. | Not performed |
| 100-180 | 32 to 150 | 40 to INC. |
| 180-250 | 17 to 66 | 10 to 41 |
| 250-297 | 5 to 40 | 9 to 26 |
| 297-425 | 10 to 30 | 8 to 30 |

The tests were also performed in seawater, with a modest, general increase in the time elapsed before complete sinking occurred. As a general rule, larger PHA microbeads sunk more quickly to the bottom.

### Example 4

### Dyeing of PHA Microbeads

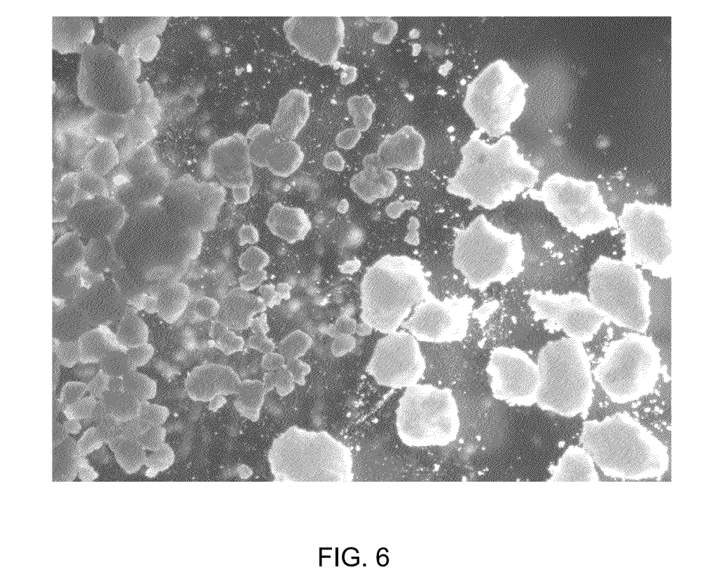

PHA microbeads of varying size and circularity were dyed with a commercially available dye. In one example, a 0.05% w/w solution (5 mL) of nile blue in ethanol was added to 1 g PHA microbeads as produced in Example 3. After waiting 48 hours, the PHA microbeads were filtered off and rinsed to yield dyed PHA microbeads. FIG. 6 shows dyed PHA microbeads on the left side of the image and undyed PHA microbeads on the right side of the image.

In a second example, a 0.05% w/w solution (5 mL) of nile blue in water was added to 1 g PHA microbeads as produced in Example 3. After waiting 48 hours, the PHA microbeads were filtered off and rinsed to yield dyed PHA microbeads.

In a third example, dyed PHA microspheres were produced by adding nile blue solution after the emulsifying step, then evaporated and filtered to produced dyed PHA microspheres.

### Example 5

### Hardness Testing of PHA used in PHA Microbeads

Mohs hardness tests were performed on PHA formulations obtained from Metabolix (Mirel™ M2100 and Mirel™ M2200) and Ecomann (Ecomann® PHA). The two Mirel™ formulations tested between 2 and 3 on the Mohs hardness scale, whereas the softer Ecomann® formulation tested between 1.5 and 2 on the Mohs hardness scale.

### Example 6

### Degradation and Sedimentation with PHA Microbeads

Various PHA microbead formulations, with different sizes and shapes, were tested to examine degradation and sequestration in seawater/sediment. In one example, 0.02 grams of 180-300 micron PHA microbeads were added to a beaker containing 40 mL of seawater with a thin layer of sediment at the bottom. The beakers were kept in the dark except for brief exposure to light on a daily basis in order to obtain a photographic image. The results are shown in FIG. 5, with images taken at days two, five, and nine (FIGS. 5A, 5B, and 5C respectively) showing progressive degradation and sequestration within the sediment. After two weeks, very little of the PHA material could be re-suspended in solution, even with gentle stirring, and most of the original PHA microbead volume was no longer visible. While some degradation had taken place within two weeks, degradation does not completely account for the removal of PHA microbeads from the sediment surface. This removal of PHA microbeads from the sediment surface reduces the likelihood that the PHA microbeads are re-suspended in an aquatic environment such as a river or an ocean.

In separate experiments conducted with varying sizes of PHA microbeads, it was determined that larger sizes of PHA microbeads (i.e., PHA microbeads greater than 400 microns in size), while sinking more quickly on average than smaller PHA microbeads, were less likely to be incorporated into the sediment on a long-term basis. Instead, PHA microbeads greater than 425 microns in size remained on the bottom but could be re-suspended in solution with modest agitation.

### Example 7

### Formulation of Lotion with PHA Microbeads

An exfoliant lotion was formulated containing PHA microbeads along with the following ingredients: deionized water, glycerin, petrolatum, distearyidimonium chloride, isopropyl palmitate, cetyl alcohol, dimethicone, allantoin, oil, benzyl alcohol. Lotions were made using PHA microbeads having the following size ranges as prepared according to Example 3: 45-100 microns, 100-180 microns, and 250-300 microns. The lotions were compared to a commercial exfoliating lotion containing polyethylene microspheres. The PHA formulations with the largest beads felt quite gritty, while the formulations with the smallest beads were deemed by testers as insufficiently abrasive. However, the formulation with PHA microbeads of 100-180 microns was deemed excellent. It should be noted that altering the hardness or shape of the PHA microbeads could allow one to develop a comparable formulation using smaller or larger PHA microbeads.

### Example 8

### Comparison of Settling Rates of Different PHA Microbeads

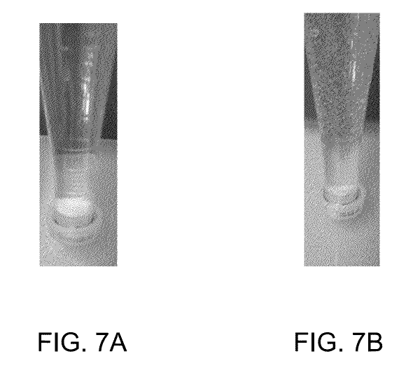

PHA microbeads produced in Example 3 were subjected to standard settling tests conducted according to the Standard Methods for the Examination of Water and Wastewater, Section 2540F. The PHA microbead samples were (i) PHA microbeads made from Ecomann® PHA powder, sized from approximately 297 μm to 425 μm, (ii) PHA microbeads made from Ecomann® PHA powder, sized from approximately 45 μm to 100 μm, and (iii) PHA microbeads made from Mirel™ pellets as described in Example 3. PHA microbeads were added to the Imhoff cone, which contained distilled water, with modest stirring. At the conclusion of the one hour test, all visible PHA microbeads from the largest size range (297 μm to 425 μm) had sunk to the bottom of the Imhoff cone, as shown in FIG. 7A.

In contrast, while a substantial portion of PHA microbeads from the smallest size range (297 μm to 425 μm) had sunk to the bottom of the Imhoff cone, a significant fraction of the PHA microbeads remained suspended in solution, as shown in FIG. 7B, wherein the suspension is cloudy compared to the image in FIG. 7A. None of this material remained on the surface of the water, but a significant fraction, approximately 30% by volume, did not sink all the way to the bottom by the conclusion of the one hour test period.

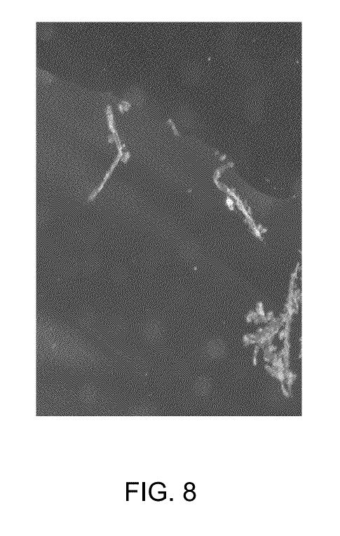

The third sample was obtained by blending Mirel™ pellets and fractionating by size. The consistency of circularity of the resulting PHA microbeads was poor, and some microbeads had a very angular shape. When subjected to the settling test described above, by the conclusion of the test, almost all visible PHA microbeads had either sunk to the bottom of the cone or were floating on the surface of the water. A significant fraction, approximately 10 percent by volume, remained floating on the top surface of the water, a very small fraction remained suspended in the water, and the bulk of the material (close to 90 percent) had sunk to the bottom of the Imhoff cone. Microscopic images were taken to compare the PHA microbeads that floated to the PHA microbeads that sunk. The floating PHA microbeads had much lower circularity than the PHA microbeads that sunk. These floating PHA microbeads (see image in FIG. 8) are likely to have a much longer residence period (relative to PHA microbeads that sink) in the environment prior to degrading. During this time, the floating particles can serve as carriers of toxic organic compounds, and can be ingested by aquatic organisms. Such PHA microbeads are disfavored according to the methods of the invention, and PHA formulations containing greater than five (5) percent of such floating PHA microbeads are not suitable according to the methods of the invention.

### INCORPORATION BY REFERENCE

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

The articles “a” and “an” are used herein to refer to one or to more than one (i.e. to at least one) of the grammatical object of the article. By way of example, “a microbead” means one microbead or more than one microbead.

Any ranges cited herein are inclusive, e.g., “between five percent and seventy-five percent” includes percentages of 5% and 75%.

### REFERENCES

Andrady, A. L. 2011. Microplastics in the marine environment. Marine Pollution Bulletin 62: 1596-1605.

Boerger, C., G. Lattin, S. L. Moore, and C. J. Moore. 2010. Plastic ingestion by planktivorous fishes in the North Pacific Central Gyre. Marine Pollution Bulletin 60: 2275-2278.

Browne, M. A., T. Galloway, and R. Thompson. 2007. Microplastic—an emerging contaminant of potential concern? Integrated Environmental Assessment and Management 3 (4): 559-566.

Browne, M. A., P. Crump, S. J. Niven, E. Teuten, A. Tonkin, T. Galloway, and R. Thompson. 2011. Accumulation of microplastic on shorelines worldwide: Sources and Sinks. Environmental Science & Technology 45: 9175-9179.

Chanprateep, S. 2010. Current trends in biodegradable polyhydroxyalkanoates. Journal of Bioscience and bioengineering 110 (6): 621-632.

Chen, G.-Q. 2010. Plastics completely synthesized by bacteria: Polyhydroxyalkanoates. In Plastics from Bacteria: Natural Functions and Applications, G.-Q. Chen (ed.), Microbiology Monographs Vol. 14. Springer-Verlag Berlin Heidelberg.

Cole, M., P. Lindeque, C. Halsband, and T. S. Galloway. 2011. Microplastics as contaminants in the marine environment: A review. Marine Pollution Bulletin 62: 2588-2597.

Costa, M. F., J. A. Ivar do Sul, J. S. Silva-Cavalcanti, M. C. B. Araujo, A. Spengler, and P. S. Tourinho. 2010. On the importance of size of plastic fragments and pellets on the strandline: A snapshot of a Brazilian beach. Environmental Monitoring and Assessment 168: 299-304.

EPA. 2003. Wastewater Technology Fact Sheet. United States Environmental Protection Agency, Office of Water, EPA 832-F-03-011.

Farrell, P. and K. Nelson. 2013. Trophic level transfer of microplastic: Mytilus edulis (L.) to Carinus maenas (L.). Environmental Pollution 177: 1-3.

Fendall, L. S. and M. A. Sewell. 2009. Contributing to marine pollution by washing your face: Microplastics in facial cleansers. Marine Pollution Bulletin 58:1225-1228.

Fossi, M. C., C. Panti, C. Guerranti, D. Coppola, M. Giannetti, L. Marsili, and R. Minutoli. 2012. Are baleen whales exposed to the threat of microplastics? A case study of the Mediterranean fin whale (Balaenoptera physalus). Marine Pollution Bulletin 64: 2374-2379.

Grage, K., A. C. Jahns, N. Parlane, R. Palanisamy, I. A. Rasiah, J. A. Atwood, and B. H. A. Rehm. 2009. Bacterial polyhdroxyalkanoate granules: Biogenesis, structure, and potential use as nano-/micro-beads in biotechnological and biomedical applications. Biomacromolecules 10: 660-669.

Gregory, M. R. 1996. Plastic ‘scrubbers’ in hand cleansers: A further (and minor) source for marine pollution identified. Marine Pollution Bulletin 32(12): 867-871.

Jardim, J. J., L. S. Alves, and M. Maltz. 2009. The history and global market of oral home-care products. Brazilian Oral Research 23(Spec. Iss 1): 17-22.

Jendrossek, D. and Handrick, R. 2002. Microbial degradation of polyhydroxyalkanoates. Annu. Rev. Microbiol. 56: 403-432.

Kershaw, P. J. and H. Leslie (eds). 2012. GESAMP working group 40. Sources, fate, & effects of micro-plastics in the marine environment—a global assessment: Report of the Inception Meeting, 13-15 Mar. 2012, UNESCO-IOC, Paris, 45 pp.

Liebezeit, G. and F. Dubaish. 2012. Microplastics in beaches of the East Frisian Islands Spiekeroog and Kachelotplate. Bull. Environ. Contam. Toxicol. 89: 213-217.

Numata, K., H. Abe, and T. Iwata. 2009. Biodegradability of poly(hydroxalkanoate) materials. Materials 2: 1104-1126.

Roy, P. K., M. Hakkarainen, I. K. Varma, and A-C. Albertsson. 2011. Degradable polyethylene: Fantasy or Reality. Environmental Science & Technology 45: 4217-4227.

Siparsky, G., K. J. Voorhees, and F. Miao. 1998. Hydrolysis of polylactic acid (PLA) and polycaprolactone (PCL) in aqueous acetonitrile solutions: Autocatalysis. Journal of Environmental Polymer Degradation 6 (1): 1-41.

Teuten et al. 2009. Transport and release of chemicals from plastics to the environment and to wildlife. Phil. Trans. R. Soc. B 2009 364, 2027-2045.

We claim:

1. A method for reducing microplastic pollution in waste streams comprising:
applying a personal care formulation to a human body; and
rinsing said formulation off of the human body with water,
wherein the rinsewater from said rinsing step ultimately drains into an aquatic environment selected from the group consisting of a wastewater treatment facility, a septic system, a stormwater system, a sewer system, a surface waterbody, and a groundwater aquatic environment; and
wherein said formulation comprises polyhydroxyalkanoate microbeads having an average size of less than 400 microns.

2. The method of claim 1, wherein at least 95% of the volume of said polyhydroxyalkanoate microbeads sinks below the water surface level at the one hour conclusion of a standard settling test conducted according to the Standard Methods for the Examination of Water and Wastewater, Section 2540F.

3. The method of claim 1, wherein at least 90 percent of the monomer units of said polyhydroxyalkanoate microbeads are hydroxybutyrate units.

4. The method of claim 1, wherein at least 25% of said polyhydroxyalkanoate microbeads are sequestered into sediment at the bottom of said aquatic environment within 10 days of said polyhydroxyalkanoate microbeads being drained into said aquatic environment.

5. The method of claim 1, wherein said polyhydroxyalkanoate microbeads have an average circularity of greater than 0.95.

6. The method of claim 1, wherein said polyhydroxyalkanoate microbeads have an average circularity of greater than 0.35 and less than 0.75.

7. The method of claim 1, wherein said polyhydroxyalkanoate microbeads have an average hardness of between 0.5 and 3.5 on the Mohs scale.

8. The method of claim 2, wherein at least 25% of the volume of said polyhydroxyalkanoate microbeads settle to the bottom at the one hour conclusion of a standard settling test conducted according to the Standard Methods for the Examination of Water and Wastewater, Section 2540F.

9. The method of claim 2, wherein at least 95% of the volume of said polyhydroxyalkanoate microbeads settles to the bottom at the one hour conclusion of a standard settling test conducted according to the Standard Methods for the Examination of Water and Wastewater, Section 2540F.

10. The method of claim 1, wherein said polyhydroxyalkanoate microbeads additionally comprise a coloring agent.

11. A method for reducing microplastic pollution in waste streams comprising:
formulating a personal care product useful for cleaning or protecting human body surfaces, and
directing users to apply said product to human body surfaces to clean said body surfaces;
wherein said product is rinsed by said users and ultimately drains into an aquatic environment selected from the group consisting of a wastewater treatment facility, a septic system, a stormwater system, a sewer system, a surface waterbody, and a groundwater aquatic environment; and
wherein said product comprises polyhydroxyalkanoate microbeads having an average size of less than 400 microns.

12. The method of claim 11, wherein at least 95% of said polyhydroxyalkanoate microbeads sink below the water surface level at the one hour conclusion of a standard settling test conducted according to the Standard Methods for the Examination of Water and Wastewater, Section 2540F.

13. The method of claim 11, wherein at least 90 percent of the monomer units of said polyhydroxyalkanoate microbeads are hydroxybutyrate units.

14. The method of claim 11, wherein at least 25% of said polyhydroxyalkanoate microbeads are sequestered into sediment at the bottom of said aquatic environment within 10 days of said polyhydroxyalkanoate microbeads being drained into said aquatic environment.

15. The method of claim 11, wherein said polyhydroxyalkanoate microbeads have an average hardness of between 0.5 and 3.5 on the Mohs scale.

16. The method of claim 12, wherein at least 95% of the volume of said polyhydroxyalkanoate microbeads settles to the bottom at the one hour conclusion of a standard settling test conducted according to the Standard Methods for the Examination of Water and Wastewater, Section 2540F.

17. The method of claim 11, wherein said polyhydroxyalkanoate microbeads have an average circularity of greater than 0.35 and less than 0.75.

18. The method of claim 11, wherein said personal care product is toothpaste.

19. The method of claim 11, wherein said personal care product is an exfoliating product.

20. The method of claim 11, wherein said personal care product is a soap bar.
